(19) United States
(12) Patent Application Publication
(10) Pub. No.: US 2006/0120906 A1
(43) Pub. Date: Jun. 8, 2006

(54) Structure and the manufacturing method for friction parts on concrete pump

(76) Inventor: LackSu Han, Pyungtaek-Si (KR)

Correspondence Address: MILES & STOCKBRIDGE PC, 1751 PINNACLE DRIVE, SUITE 500, MCLEAN, VA, 22102-3833, US

(21) Appl. No.: 10/540,555

(22) PCT Filed: Jan. 13, 2003
(86) PCT No.: PCT/KR03/00062

§ 371 Date: Jun. 24, 2005

(30) Foreign Application Priority Data
Dec. 24, 2002 (KR) 10 2002 0083789

(51) Int. Cl. A61M 1/00

(52) U.S. Cl. 417/437

(57) ABSTRACT

The present invention relates to a sliding motion structure for a concrete pump that can reduce the extent of abrasion and solve the problem of high maintenance expenses resulting from frequency replacement of the sliding motion structure by fabricating friction areas with a plurality of wear-resistant friction blocks, and to a manufacturing method thereof. Also, the sliding motion structure and the manufacturing method thereof can also prevent the internal wall surfaces of the inlet ports and outputs, through which concrete is transferred, from being worn out and prevent the external end of the connecting pipe from being worn out partially. The sliding motion structure of the present research includes a plane fixed member having a wear plate, a pair of coupling tubes, first friction members, and a second friction member; and a ring-shaped movable member having a ring-shaped connecting pipe and a third friction member which is formed of tungsten carbide and connected to the first and second friction members tightly along the ends of the connecting pipe in the same length.

### TECHNICAL FIELD

The present invention relates to a concrete pump; and, more particularly, to a sliding motion structure for use in a concrete pump and a method for manufacturing the same, wherein the sliding motion structure is capable of solving a problem of high maintenance expenses resulting from frequent replacement thereof as well as preventing the internal walls of inlet/outlet ports inputting/outputting a concrete being worn out and the external end of a coupling pipe from being unevenly worn out by fabricating friction areas of the sliding motion structure with a plurality of wear-resistant friction blocks, thereby reducing a wear rate thereof.

### BACKGROUND ART

FIG. 1 is a perspective view illustrating one example of conventional concrete pumps, as shown, a pair of concrete cylinders 12a and 12b, wherein concrete pistons 11a and 11b are installed therein, respectively, and a plane fixed member 13 having a pair of inlet ports is connected to the outlet ports of the concrete cylinders 12a and 12b, the inlet ports 13a and 13b of the fixed member 13 being connected with the inside of concrete cylinder 12a or 12b, respectively.

A ring-shaped movable member 14 is adhered tightly to the external surface of the fixed member 13 and the movable member 14 is fixed to a line end of an S-shaped gate valve 15, which is shaken by a driving cylinder 16, the movable member 14 being connected with one of the inlet ports 13a and 13b alternately by the shaking movement of the S-shaped gate valve 15.

A piston 17, which is inserted into the inside of a cylinder 18, is formed on the external circumferential surface of the rear end of the S-shaped gate valve 15 and the S-shaped gate valve 15 is connected in such a way that it can be moved along its axial direction and shaken around the axial line. A pressure chamber is formed between the piston 17 and the rear part of the cylinder 18 and the pressure chamber is connected to a tank through a hydraulic pipe 21. The hydraulic pipe 21 has a hydraulic pump 20 installed therein and a safety valve (V) is formed in a branch pipe 22 which is diverged from the hydraulic pipe 21 and connected to the tank.

The concrete pump of the above mentioned structure is operated as follows. First, the driving cylinder 16 is operated so that the movable member 14 is connected to the inlet port 13a. Then, the concrete in a concrete cylinder 12a comes out through the inlet port 13a, the movable member 14, the S-shaped gate valve 15 and a discharging pipe 19 by moving forward the concrete piston 11a in one side and, at the same time, the concrete in a hopper 10 is injected into the concrete cylinder 12b by moving backward a concrete piston 11b in the other side.

When the concrete is injected and discharged by the movement of the concrete pistons 11a and 11b, the movable member 14 is connected with the other inlet port 13b by operating the driving cylinder 16 and moving the S-shaped gate valve 15 in the other side. Subsequently, the concrete injected into the inside of the concrete cylinder 12b is discharged through the inlet port 13b, the movable member 14, the S-shaped gate valve 15 and the discharging pipe 19 by moving forward the concrete piston 11b and, at the same time, the concrete in the hopper 10 is injected into the concrete cylinder 12a through the inlet port 13a by moving backward the concrete piston 11a.

During the shaking of the S-shaped gate valve 15 and, as a result, the movable member 14 is shaken while it is tightly adhered to the fixed member 13, because the hydraulic pump 20 is operated to thereby apply strong pressure to the pressure chamber. So, the concrete does not leak between the movable member 14 and the fixed member 13. In short, the movable member 14 is shaken and rubbed strongly against the fixed member 13, thus requiring the development of a movable member and a fixed member having a low abrasion rate.

### DISCLOSURE OF INVENTION

Conventional fixed members and movable members, that is, sliding motion structures, are formed of high manganese cast-iron so they have relatively fine wear-resistance, but when they are used for about 60 days, they reach their wear threshold and should be replaced with new ones, which causes inconvenience for workers who should change the sliding motion structure frequently and, thus, require an excessive maintenance cost and causes a problem of wasting by disposing the whole fixed members and movable members.

In the conventional sliding motion structures, the internal wall surfaces of the inlet ports of the fixed member and the outlet ports of the movable member are worn seriously by being exposed to the transferred concrete, and the external end of the movable member, which is shaken along with an S-shaped gate valve, is worn partially as it is frictionized with the surface of the fixed member.

It is, therefore, an object of the present invention to provide a sliding motion structure for a concrete pump that has a low abrasion rate by forming the abrasive areas of the fixed member and the movable member with highly wear-resistant tungsten carbide and to provide a method for manufacturing the sliding motion structure and, thus, the sliding motion structure of the present invention can solve the problems of high maintenance cost and wasteful use of resources resulting from frequency replacement of the fixed member and the movable member, the sliding motion structures with long life span capable of being provided at a relatively low cost.

It is another object of the present invention to provide a sliding motion structure for a concrete pump that can reduce the cost for changing parts, i.e., the fixed member and the movable member, by making the friction members with a plurality of tungsten carbide friction blocks and changing only those worn out among the friction blocks with new friction blocks, when the parts are worn out partially, and to provide a method for manufacturing the sliding motion structure, the sliding motion structure using the friction blocks capable of preventing deformation of the wear plate caused by the difference in the thermal expansion coefficients when the tungsten carbide is welded on a different kind of metal.

It is another object of the present invention to provide a sliding motion structure for a concrete pump that can reduce the extent of abrasion against concrete by reinforcing the internal wall surfaces of the inlet port of the fixed member and the internal wall surfaces of the outlet port of the movable member, and prevents the external end of the movable member from being worn partially due to its friction against the surface of the fixed member by reinforcing the external end of the movable member, which is shaken along with the S-shaped gate valve.

In accordance with one aspect of the present invention, there is provided a sliding motion structure for a concrete pump which extrudes concrete by using concrete pistons moving back and forth in a pair of concrete cylinders and discharges the extruded concrete through a shaking S-shaped gate valve, including: a plane fixed member including a wear plate which is connected to the ends of the concrete cylinders and has a pair of throughhole portions connected to the insides of the concrete cylinders, a pair of coupling tubes which are connected with the wear plate along the throughhole portions and have inlet ports, first friction members which are formed of tungsten carbide and connected to the upper surface of the coupling tubes, and a second friction member which is formed of tungsten carbide and connected on the upper surface of the wear plate between the throughhole portions in the same height as the first friction members; and a ring-shaped movable member including a ring-shaped connecting pipe which is connected to the ends of the S-shaped gate valve and a third friction member which is formed of tungsten carbide and connected to the first and second friction members tightly along the ends of the connecting pipe in the same length.

In accordance with another aspect of the present invention, there is provided a sliding motion structure wherein the coupling tubes of the fixed member are provided with a protrusion formed downwards along the internal circumference area of the lower part of the coupling tubes to guide the coupling tubes to the upper part of the throughhole portions of the wear plates; and the coupling tubes are provided with a standard protrusion formed upwards along the external circumference area of the upper part of the coupling tubes to guide the connection between the coupling tubes and the first friction members.

In accordance with another aspect of the present invention, there is provided a sliding motion structure wherein reinforce members are inserted to the internal walls of the coupling tubes of the fixed member and the internal wall of the connecting pipe of the movable member.

In accordance with another aspect of the present invention, there is provided a sliding motion structure wherein the coupling tubes of the fixed member are provided with catching protrusions formed inwards horizontally along the internal circumference of the upper part of the coupling tubes, the catching protrusions limiting the upward insertion of the reinforce members.

In accordance with another aspect of the present invention, there is provided a sliding motion structure wherein the friction members are formed of combinations of friction blocks.

In accordance with another aspect of the present invention, there is provided a method for manufacturing a sliding motion structure for a concrete pump, the method including the steps of: a) fabricating a wear plate with a pair of throughhole portions connected to the insides of a pair of concrete cylinder; b) fabricating coupling tubes having inlet ports to be tightly inserted to the throughhole portions of the wear plate; c) connecting first friction members which are formed of tungsten carbide to the upper part of the coupling tubes; d) connecting the coupling tubes combined with the first friction members to the throughhole portions of the wear plate, the first friction members being protruded to the upper part of the wear plate; e) fabricating a second friction member with tungsten carbide and inserting the second friction member between the first friction members in the wear plate; f) connecting a connecting member to the lower part of the second friction member, the upper surfaces of the first and second friction members are on the same plane; and g) connecting the connecting member combined with the second friction member to a part of the wear plate between the first friction members, the upper surfaces of the first and second friction members are on the same plane.

### BRIEF DESCRIPTION OF DRAWINGS

The above and other objects and features of the present invention will become apparent from the following description of the preferred embodiments given in conjunction with the accompanying drawings, in which:

FIG. 1 is a perspective view illustrating a conventional concrete pump;

FIG. 2 is an exploded perspective view showing a sliding motion structure in accordance with an embodiment of the present invention;

FIG. 3 is a perspective view showing the sliding motion structure of FIG. 2;

FIG. 4 is a cross-sectional view depicting a side of the sliding motion structure of FIG. 3;

FIG. 5 is a cross-sectional view illustrating a movable member in accordance with another embodiment of the present invention;

FIG. 6 is a flowchart describing a process for manufacturing a sliding motion structure in accordance with an embodiment of the present invention;

FIG. 7 is an exploded perspective view showing a sliding motion structure in accordance with another embodiment of the present invention;

FIG. 8 is a perspective view depicting the sliding motion structure of FIG. 7;

FIG. 9 is a cross-sectional view depicting a side of the sliding motion structure of FIG. 8; and

FIG. 10 is a flowchart describing a process for manufacturing a sliding motion structure in accordance with another embodiment of the present invention.

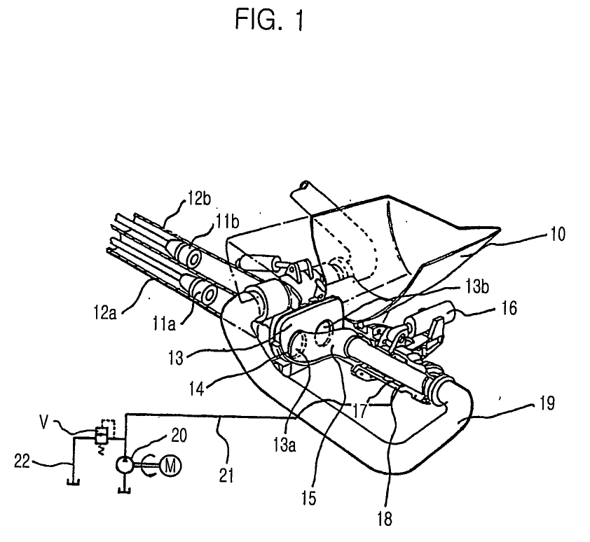

### BEST MODE FOR CARRYING OUT THE INVENTION

Other objects and aspects of the invention will become apparent from the following description of the embodiments with reference to the accompanying drawings, which is set forth hereinafter.

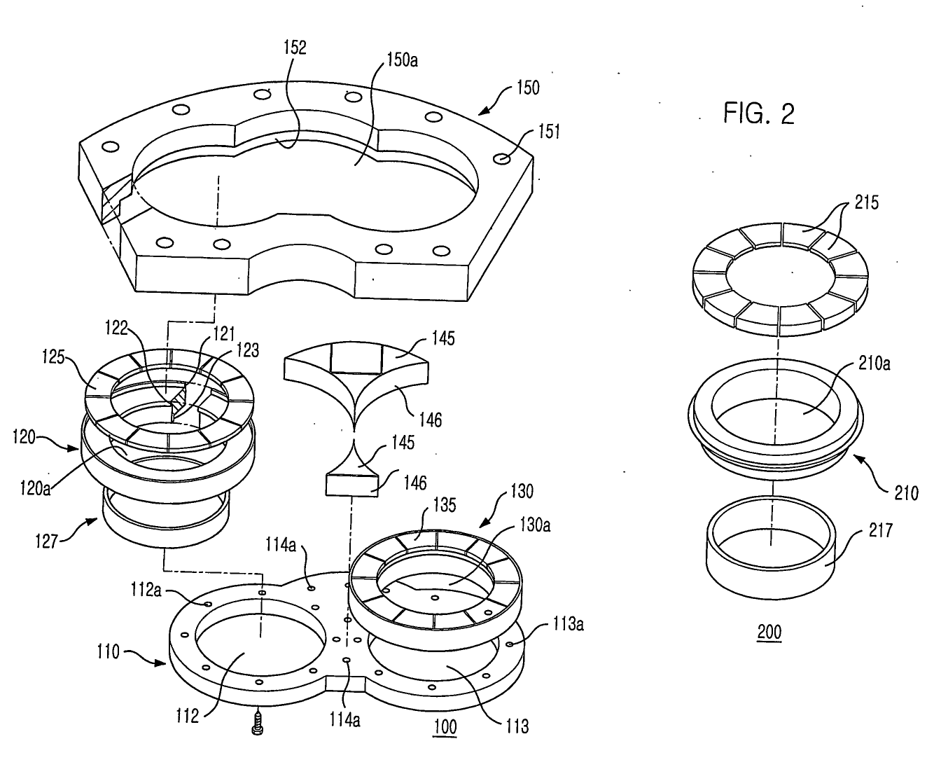

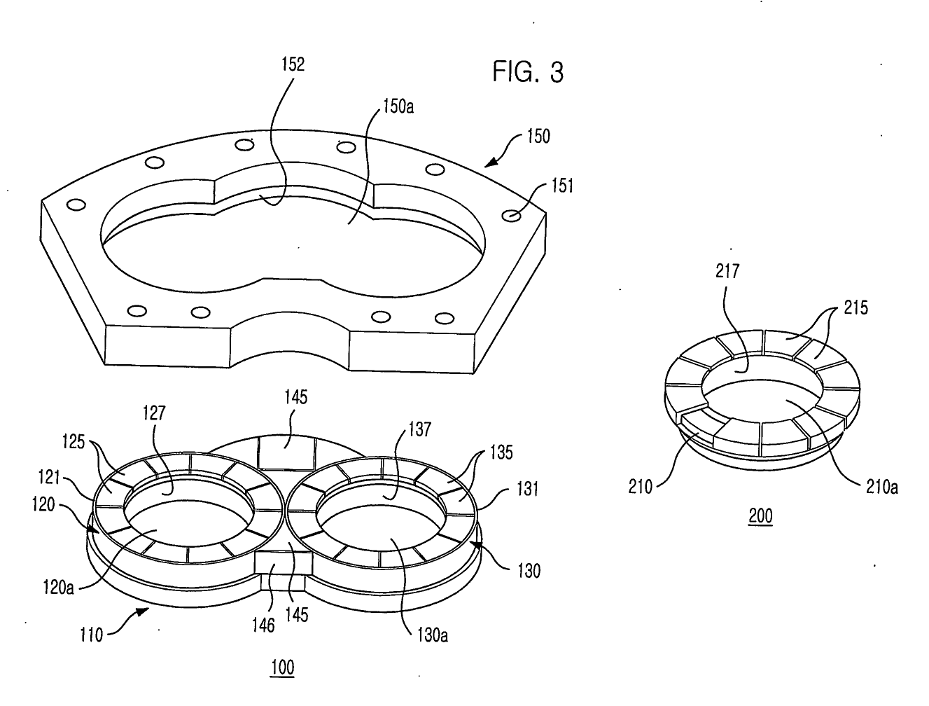

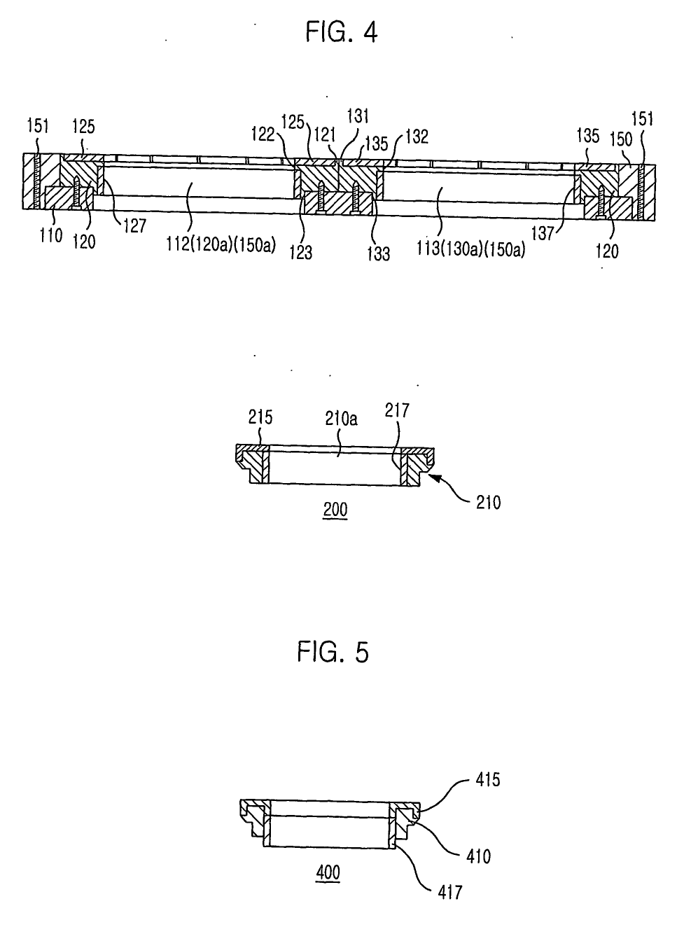

FIG. 2 is an exploded perspective view showing a sliding motion structure in accordance with an embodiment of the present invention. FIG. 3 is a perspective view showing the sliding motion structure of FIG. 2, and FIG. 4 is a cross-sectional view depicting a side of the sliding motion structure of FIG. 3. The sliding motion structure for a concrete pump includes a plane fixed member 100 having a pair of inlet ports 120a and 120b; and a ring-shaped movable member 200 having an outlet port 210a.

The concrete pump extrudes concrete by using concrete pistons that move back and forth alternately in a pair of concrete cylinders and discharges the concrete through an S-shaped gate valve in which the extruded concrete is shaken, and the fixed member 100 and the movable member 200 are fixed firmly on the ends of the concrete cylinders and the end of the S-shaped gate valve, thus the movable member 200 performing sliding back and forth according to the shaking of the S-shaped gate valve, while it is tightly adhered to the fixed member 100 and, accordingly, the outlet port 210a of the movable member 200 being connected to each of the first and second inlet ports 120a and 130a alternately.

The plane fixed member 100 includes a wear plate 110, a pair of coupling tubes 120 and 130, first friction members 125 and 135, a second friction member 145 and 150, and a holder 150.

The wear plate 110, which is a carbon steel sheet that is firmly connected with the ends of the concrete cylinders, has a pair of throughhole portions 112 and 113 that are connected to the insides of the concrete cylinders, individually. The wear plate 110 also has a plurality of bolt fixing holes for fixing the coupling tubes 120 and 130 thereon along the circumference areas of the throughhole portions 112 and 113 and, on the friction surface between the throughhole portions 112 and 113, a plurality of bolt fixing holes, i.e., second bolt fixing holes, for fixing the second friction member 135 are formed.

The coupling tubes 120 and 130 which have the inlet ports 120a and 130a penetrated in the center are inserted and tightly adhered to the throughhole portions 112 and 113, respectively. The coupling tubes 120 and 130 have insertion protrusions 123 and 133 formed downwards and inserted to the inside of the throughhole portions 112 and 113 tightly and, due to the insertion protrusions 123 and 133, the coupling tubes 120 and 130 are connected with the upper part of the throughhole portions 112 and 113 of the wear plate 110 precisely. Subsequently, the coupling tubes 120 and 130 are fixed on the upper part of the throughhole portions 112 and 113 by utilizing a plurality of bolts fixed in the lower part of the coupling tubes 120 and 130 through the bolt fixing holes 112a and 113a around the throughhole portions 112 and 113.

The first friction members 125 and 135 are fixed on the upper surfaces of the coupling tubes 120 and 130, while maintaining a predetermined space between them and, desirably, the first friction members 125 and 135 include a standard protrusion 121, which is formed upwards along the external circumference areas of the coupling tubes 120 and 130, has a predetermined thickness, and it guides the first friction members 125 and 135 to be fitted into the coupling tubes 120 and 130.

The first friction members 125 and 135, which are formed of tungsten carbide, are connected with the upper surface of the coupling tubes 120 and 130 in such a method as welding, and the first friction members 125 and 135 are formed in the same height so that the end of the movable member 200 which is connected to the S-shaped gate valve is adhered tightly.

The second friction member 145 also is formed of tungsten carbide and it is formed in the area between the throughhole portions 112 and 113 of the wear plate 110. The second friction member 145 is formed in the same height as the first friction members 125 and 135 which are formed on the wear plate 110 and, thus, it forms a friction surface that is frictionized against the end of the movable member 200 during the shaking movement of the S-shaped gate valve.

If the second friction member 145 is fixed directly on the friction surface of the wear plate 110 in such a method as welding, the wear plate 110 may be deformed due to the difference in the thermal expansion coefficients. Therefore, it is desirable to connect the second fraction unit 145 with the wear plate 110 by fixing a metallic connecting member 146 on the lower part of the second fraction unit 145 in the welding method and utilizing the bolts that penetrate the second bolt fixing holes 114a of the wear plate and connected with the connecting member 146.

Since the carbon steel that forms the wear plate 110, the coupling tubes 120 and 130, and the connecting member 146 has a thermal expansion coefficient of 14×10⁻⁶/° C., while the tungsten carbide that forms the first and second friction members 125, 135 and 145 has a thermal expansion coefficient of 3×10⁻⁶/° C., if the first and second friction members 125, 135 and 145 are formed in an integrated form, they 125, 135 and 145 connected with the coupling tubes 120 and 130 and the connecting member 146 come off when the surface temperature is changed and, in this case, the concrete may be leaked. Therefore, it is desirable to fabricate each of the fraction units 125, 135 and 145 with a plurality of friction blocks, and the use of friction blocks can minimize the differences in the thermal expansion coefficients between the coupling tubes 120 and 130, the connecting member 146, and the friction members 125, 135 and 145 and, thus, the friction members 125, 135 and 145 do not come off from the coupling tubes 120 and 130 and the connecting member 146.

Reinforce members 127 and 137 are tightly adhered to the internal wall surfaces of the coupling tubes 120 and 130, respectively, to thereby reinforce the internal wall surfaces which are frictionized hard against the transferred concrete. The reinforce members 127 and 137 may be tightly adhered to the internal wall surfaces of the coupling tubes 120 and 130 by welding the first friction members 125 and 135 to the coupling tubes 120 and 130, shrink-fitting the reinforce members 127 and 137 into the heated coupling tubes 120 and 130 with force right after the welding, and welding the adhesion area.

If the reinforce members 127 and 137 are shrink-fitted into the internal wall surfaces of the coupling tubes 120 and 130 with force as described in the above, by installing the linkage protrusion 122 extended in the axial direction in a predetermined width along the internal circumference area of the upper surface of the coupling tubes 120 and 130, each of the reinforce members 127 and 137 inserted and coupled from the lower part of the internal wall surface of the coupling tubes 120 and 130 to the upper part is inserted only to a predetermined height in each of the internal wall surface of the coupling tubes 120 and 130. In short, the setup locations of the reinforce members 127 and 137 are controlled precisely.

Diverse kinds of alloys can be used to form the reinforce members 127 and 137, for example, hi-chrome steel containing about 20% tungsten carbide can be used to form the reinforce members 127 and 137.

The holder 150 connects the wear plate 110, which fixes the first and second friction members 125, 135 and 145, the coupling tubes 120 and 130, and the connecting member 146 thereon, with the ends of the concrete cylinders, and the holder 150 is formed in the shape of a frame in which a throughhole member 150a, through which part of the first and second friction members 125, 135 and 145 penetrate, is made in the center, and along the external circumference of the holder 150, a plurality of bolt fixing holes for fixing bolts that penetrate the fringe part of the ends of the concrete cylinders are formed. On the internal wall surface of the throughhole member 150a, a catching hump 152 is formed to catch the circumference area of the wear plate 110 being inserted to the throughhole member 150a.

The ring-shaped movable member 200, which is connected with the end of the S-shaped gate valve, includes a connecting pipe 210 and a third friction member 215.

The connecting pipe 210 is formed of carbon steel and it is formed in the shape of a ring having an outlet port 210a in the center. To the end of the connecting pipe 210, the third friction member 215 formed of tungsten carbide is connected in the same height as the third friction member 215, and the third friction member 215 is tightly adhered to the friction members 125, 135 and 145 of the fixed member 100 while is shaken along with the connecting pipe 210 during the shaking movement of the S-shaped gate value.

Also, it is desirable to fabricate the third friction member 215 with a combination of friction blocks to minimize the difference in the thermal expansion rates, and when the third friction member 215 is fabricated, each of the friction blocks is fixed firmly by being welded on the end of the connecting pipe 210. It is also possible to have the first reinforce member 217 tightly adhered to the internal wall surface of the connecting pipe 210 in consideration of the friction against the concrete transferred through the connecting pipe 210.

The third friction member 215 of the movable member 200 can be formed in a planar shape, but it is desirable to form it in the shape of [symbol] as illustrated in FIGS. 2 to 4, to effectively prevent the external circumference area from being worn out by the repeated friction, that is, the external end of the third friction member 215 is bent downwards. It is also desirable to form groove along the circumference area at the end of the connecting pipe 210 so that the third friction member 215 has a cross section of

[symbol]

FIG. 5, which is a cross-sectional view illustrating the third friction member connected with the movable member 400 in accordance with another embodiment of the present invention, shows the fiction blocks bent downwards as well as the external ends to thereby have a cross section of [symbol] This structure can prevent the internal end of the third friction member 415 as well as its external end from being partially worn out, effectively.

In FIG. 5, the lower part of the reinforce member 417 is protruded to the lower part of a connecting pipe 410 in consideration of a case where the movable member 400 connected with the S-shaped gate value requires a three-step shape, and If the movable member 400 connected with the S-shaped gate value requires a two-step shape, the reinforce member 417 is formed short not to be protruded to the lower part of the connecting pipe 410.

Meanwhile, the tungsten carbide used to form the friction members in the present invention is prepared as metal mixture containing 80 to 90% WC, 2 to 25% TiC, 3 to 10% TaC, and 3 to 10% TaNBC to thereby have a wear-resistant over 90 HRC.

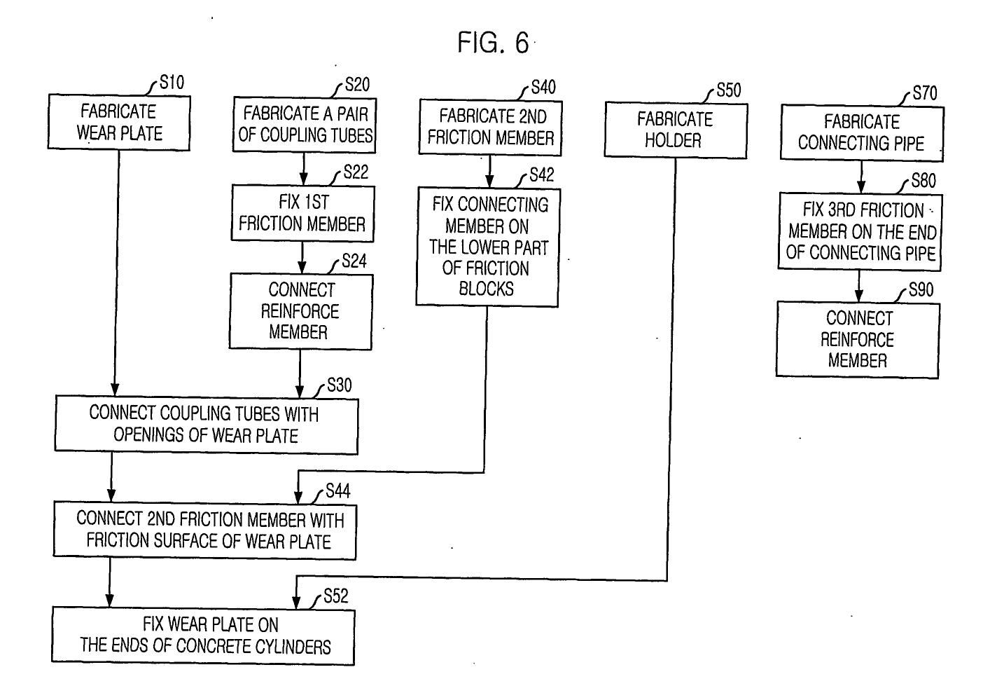

A process for manufacturing a sliding motion structure having the above described elements, as shown in FIG. 6, includes a steps of fabricating a wear plate (S20), fabricating coupling tubes (S20), fixing first friction members (S22), connecting the coupling tubes (S30), fabricating a second friction member (S40), connecting a connecting part (S42), fixing a second friction member (S44), fabricating a holder (S50), and installing the fixed member.

At step S10, the wear plate 110 connected with the ends of a pair of concrete cylinders is fabricated, and the wear plate 110 has a pair of throughhole portions 112 and 113 to be connected with the insides of the concrete cylinders on both right and left sides, and on the circumference area and friction surface of the wear plate 110, a plurality of bolt fixing holes 112a, 113a and 114a are penetrated.

At step S20, a pair of coupling tubes 120 and 130 are fabricated. The coupling tubes 120 and 130 which have inlet ports 120a and 130a in the center are formed to be closely connected to the upper surface of the throughhole portions 112 and 113. The coupling tubes 120 and 130 also have protrusions 123 and 133 to be closely inserted to the insides of the throughhole portions 112 and 113 which are formed downwards along the internal circumference area of the lower part of the coupling tubes 120 and 130 and the protrusions 123 and 133 help the coupling tubes 120 and 130 to be connected to the upper part of the throughhole portions 112 and 113 closely.

It is desirable to form a reference protrusion 121 formed upwards to guide the first friction members 125 and 135 to their connection location easily, the reference protrusion 121 having a predetermined thickness along the external circumference area of the upper surface of the coupling tubes 120 and 130.

After the coupling tube fabrication step S20, the step S22 wherein the first friction member is fixed is executed. At step S22, the first friction members 125 and 135 formed of tungsten carbide are fixed on the upper surface of the coupling tubes 120 and 130 in such a method as welding. The first friction members 125 and 135 are made with a plurality of friction blocks and welded along the upper surface of the coupling tubes 120 and 130.

Subsequently, at step S30, the coupling tubes 120 and 130 combined with the first friction members 125 and 135 are connected with the throughhole portions 112 and 113 of the wear plate 110. The protrusions 123 and 133 formed downwards along the internal circumference area of the lower surface of the coupling tubes 120 and 130 are inserted to the throughhole portions 112 and 113 of the wear plate 110, and the coupling tubes 120 and 130 are firmly fixed on the upper part of the throughhole portions 112 and 113 by putting the coupling tubes 120 and 130 on the upper surfaces of the throughhole portions 112 and 113, fixing them with a plurality of bolts inserted to the bolt fixing holes 112a and 113a around the throughhole portions on the lower part of the coupling tubes 120 and 130.

At step S40, a second friction member 145 is fabricated to fill the friction surface between the throughhole portions 112 and 113 of the wear plate 110 and the second friction member 145 is fabricated with a plurality of friction blocks to prevents thermal deformation. Subsequently, at step S42, the connecting member 146 is welded on the lower part of the friction blocks that constitute the second friction member 145. When the second friction member 145 welded with the connecting member 146 is filled in the friction surface, the upper surface of the second friction member 145 is no higher than the first friction members 125 and 135.

At step S44, the second friction member 145 with the connecting member 146 is fixed on the friction surface of the wear plate 110 by using a plurality of bolts penetrating the bolt fixing holes 114a formed on the friction surface and fixed on the lower part of the connecting member 146 and, as a result, the first friction members 125 and 135 and the second friction member 145 are formed in the same plane on the upper part of the wear plate 110.

At step S50, the holder 150 for connecting the wear plate 110 with the ends of the concrete cylinders are fabricated. The wear plate 110 is already combined with the first and second friction members 125, 135 and 145, the coupling tubes 120 and 130 and the connecting member 146. The holder 150 has a throughhole member 150a through which the first and second friction members 125, 135 and 145 penetrate in the center; a plurality of bolt fixing holes 151 around the external circumference of the holder 150; and a catching hump 152 formed along the internal wall surface of the throughhole member 150a.

At step S52, the wear plate 110, which is combined with the first and second friction members 125, 135 and 145, the coupling tubes 120 and 130, and the connecting member 146, is connected with the ends of the concrete cylinders by utilizing the holder 150. The first and second friction members 125, 135 and 145 are inserted to the holder 150 through the throughhole member 150a and, when the circumference area of the wear plate 110 is caught by the catching hump 152 of the holder 150, the bolts are fixed into the bolt fixing holes 151 of the holder 150, penetrating the bolt fixing holes on the fringe of the ends of the concrete cylinders. This way, the fixed member 100 is firmly connected with the ends of the concrete cylinders.

Between the step S22 for fixing the first friction member and the step S30 for fixing the coupling tubes, a step S24 for providing reinforcement can be added. At the step S24, reinforce members 127 and 137 are tightly connected to the internal wall surface of the coupling tubes 120 and 130. That is, the first friction members 125 and 135 are welded on the upper part of the coupling tubes 120 and 130 and right after the welding, the reinforce members 127 and 137 are inserted with force to the internal wall surfaces of the coupling tubes 320 and 330 in the method of shrink fitting, while the coupling tubes 120 and 130 are heated. In order to maximize the coherence between the reinforce members 127 and 137 and the coupling tubes 120 and 130, it is desirable to weld the circumference area of the connection between the reinforce members 127 and 137 and the coupling tubes 120 and 130, and through the above series of processes, the fabrication of the fixed member 100 is completed.

Meanwhile, the movable member 200 is fabricated through the processes of forming a connecting pipe, fixing a third friction member, and connecting a reinforce member.

The fabrication of the movable member 200 is carried out through the steps of fabricating connecting pipe 210 (S70), fixing the third friction member (S80), and connecting the reinforce member (S90).

At step S70, a ring-shaped connecting pipe 210 to be connected with the end of the S-shaped gate valve is fabricated and, at step S80, the third friction member 215 formed of tungsten carbide is welded on the end of the connecting pipe 210. The external end of the third friction member 215 can be bent downwards and the third friction member 215 is formed of a plurality of friction blocks, each having a cross section of a ‘ [symbol] ’ shape and, in this case, the connecting pipe 210 is formed to have a subsided fringe along the external circumference area at the end.

Right after the third friction member 215 is welded on the connecting pipe 210, at step S90, the reinforce member 217 is inserted to the internal wall surfaces of the coupling tubes 320 and 330 in the method of shrink fitting while the connecting pipe 210 is heated. To enhance the coherence between the reinforce member 217 and the connecting pipe 210 after the connection, it is desirable to weld connection area between the reinforce member 217 and the connecting pipe 210.

The concrete pump formed by connecting the fixed member 100 and the movable member 200 is operated as follows.

When a pair of concrete cylinders perform an alternate back and forth movement to discharge concrete, the S-shaped gate valve is shaken while pressure is applied thereto, and during the shaking, the fixed member 100 and the movable member 200 are frictionized against each other strongly. According to the present invention, since the friction occurs only between the wear-resistant first and second friction members 125, 135 and 145, the extent of abrasion caused by the friction against the concrete is reduced remarkably. Therefore, the fixed member 100 and the movable member 200 can be used for a long time and, thus, the delay in work process which is caused by frequent replacement of the fixed member 100 and the movable member 200 can be reduced. Moreover, when the fixed member 100 and the movable member 200 need to be replaced after the long-time use, only those worn out among the friction blocks constituting the first to third friction members 125, 135, 145 and 215 are replaced with new friction blocks. Therefore, it becomes easy and inexpensive to maintain the concrete pump.

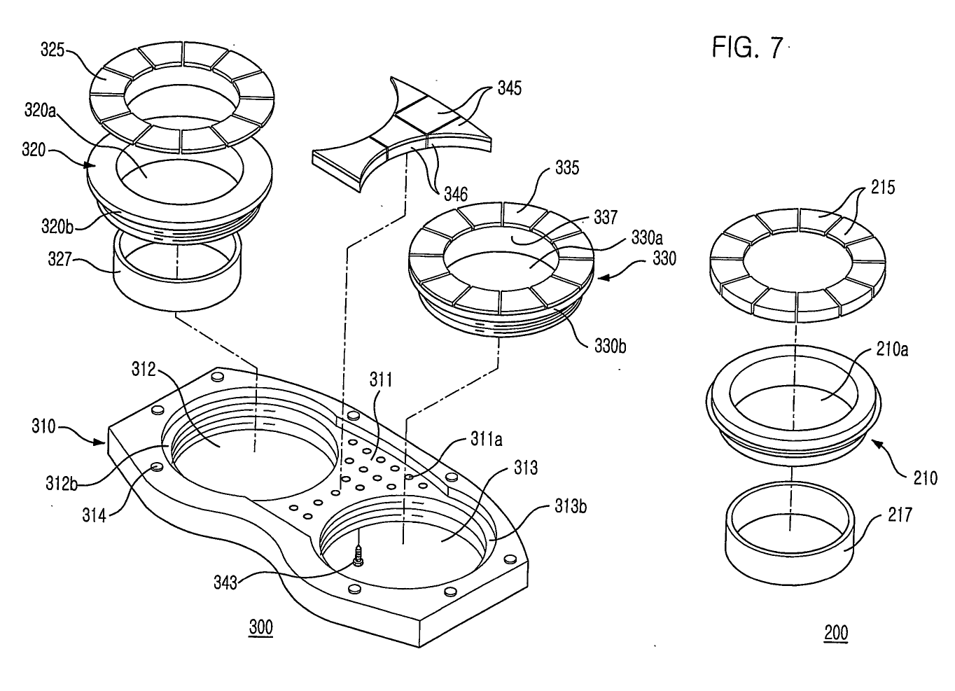

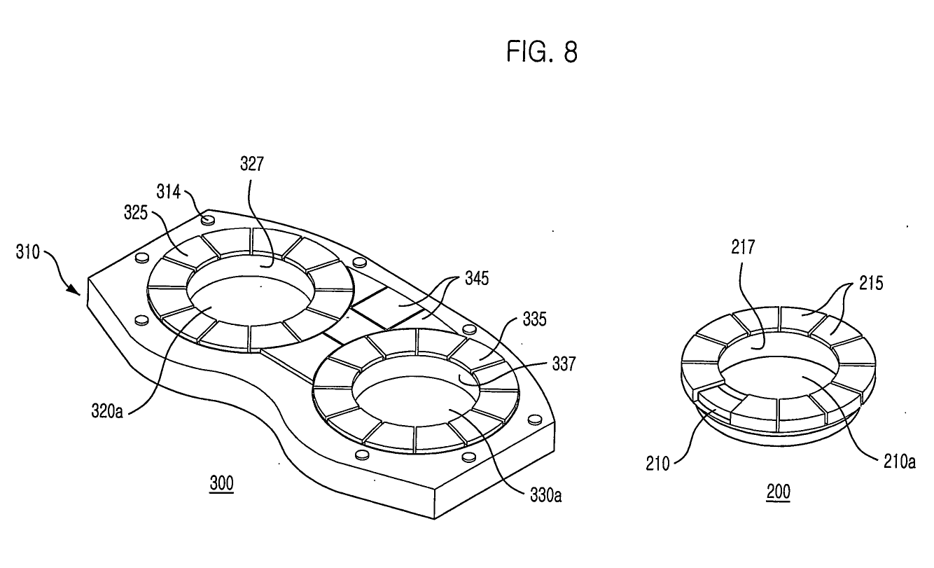

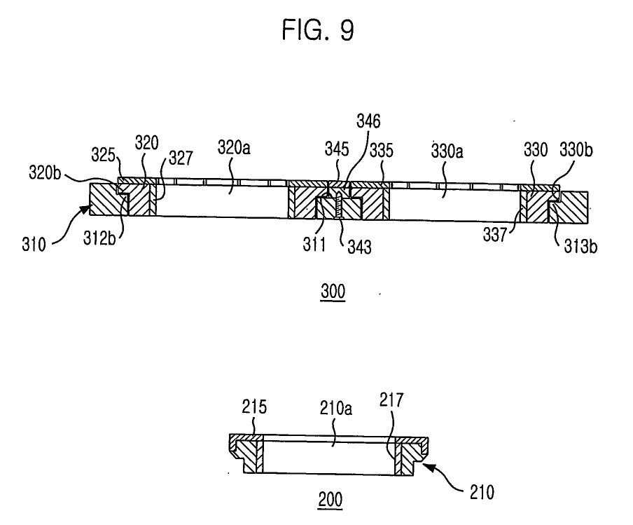

FIG. 7 is an exploded perspective view showing a sliding motion structure for a concrete pump in accordance with another embodiment of the present invention; FIG. 8 is a perspective view depicting the sliding motion structure of FIG. 7; and FIG. 9 is a cross-sectional view depicting a side of the sliding motion structure of FIG. 8. As shown in the drawing, the sliding motion structure for the concrete pump includes a plane fixed member 300 and a ring-shaped movable member 200. Since the movable member 200 is the same that is described in the previous embodiment described earlier, further description on it will be omitted herein.

The plane fixed member 300 includes a wear plate 310, a pair of coupling tubes 320 and 330, first friction members 325 and 335, and second friction members 345.

The wear plate 310, which is a carbon steel firmly connected with the ends of a pair of concrete cylinders, has a pair of throughhole portions 312 and 313 connected to the insides of the concrete cylinders on the right and left and on the friction surface between the throughhole portions 312 and 313, a subsidence 311 is formed in a predetermined depth and also bolt fixing holes 314 for connecting the wear plate 310 with the ends of the concrete cylinders are formed along the circumference of the wear plate 310.

The coupling tubes 320 and 330 are rings having inlet ports 320a and 330a in the center. They are formed to be connected with the throughhole portions 312 and 313 of the wear plate 310 with bolts. For the connection, a bolt fixing area is formed on the external wall surfaces 320b and 330b of the coupling tubes 320 and 330 and on the internal wall surface of the throughhole portions 312 and 313. The coupling tubes 320 and 330 can also be combined with the throughhole portions 312 and 313 of the wear plate 310 in the method of tight adhesion, instead of using bolts.

The coupling tubes 320 and 330 are inserted from the lower part of the wear plate 310 into predetermined positions of the throughhole portions tightly by forming subsided fringes 312b and 313b in the upper part of the internal wall of the throughhole portions 312 and 313 of the wear plate 310 and forming protruded fringes 320b and 330b which are inserted into the subsided fringes 312b and 313b tightly on the upper part of the external wall of the coupling tubes 320 and 330. The connection between the coupling tubes 320 and 330 and the wear plate 310 is made firmly by utilizing bolts.

The first friction members 325 and 335 which are formed of tungsten carbide are fixed on the upper part of the coupling tubes 320 and 330 in the method of welding, and when the coupling tubes 320 and 330 are inserted to the insides of the throughhole portions 312 and 313, the first friction members 325 and 335 are connected to be protruded from the surface of the wear plate 310 to a predetermined length and, as a result, the first friction members 325 and 335 can be adhered tightly to the end of the movable member 200 connected with the S-shaped gate valve.

The second friction member 345, which is also formed of tungsten carbide, is set on the subsidence 311 of the wear plate 310 to have the same height of the first friction members 325 and 335. The second friction member 345 provides a friction surface to be frictionized against the end of the movable member 200, when the S-shaped gate valve is shaken.

If the second friction member 345 is fixed on the subsidence 311 of the wear plate 310 directly in such a method as a welding, the wear plate 310 may be deformed. Therefore, it is desirable to connect a metallic connecting member 346 to the lower part of the second friction member 345 by a method such as a welding and connect the wear plate 310 to the connecting member 346 combined with the second friction member 345 by penetrating the bolts 343 through the subsidence 311 of the wear plate 310.

It is desirable to make the first and second friction members 325, 335 and 345 with a plurality of friction blocks in order to prevent the wear plate 310, coupling tubes 320 and 330 and connecting member 346 that are formed of carbon steel and the first and second friction members 325, 335 and 345 that are formed of tungsten carbide from coming off due to the difference in the thermal expansion coefficients.

The internal wall surfaces of the coupling tubes 320 and 330, which are exposed to concrete, are reinforced with reinforce members 327 and 337. The reinforce members 327 and 337 are tightly adhered to the internal wall surfaces of the coupling tubes 320 and 330. The reinforce members 327 and 337 are shrink-fitted into the coupling tubes 320 and 330 with force right after the first friction members 325 and 335 are welded onto the coupling tubes 320 and 330 when the coupling tubes 320 and 330 are still heated, and then the adhesion areas are welded so that the reinforce members 327 and 337 are tightly adhered to the internal wall surfaces of the coupling tubes 320 and 330.

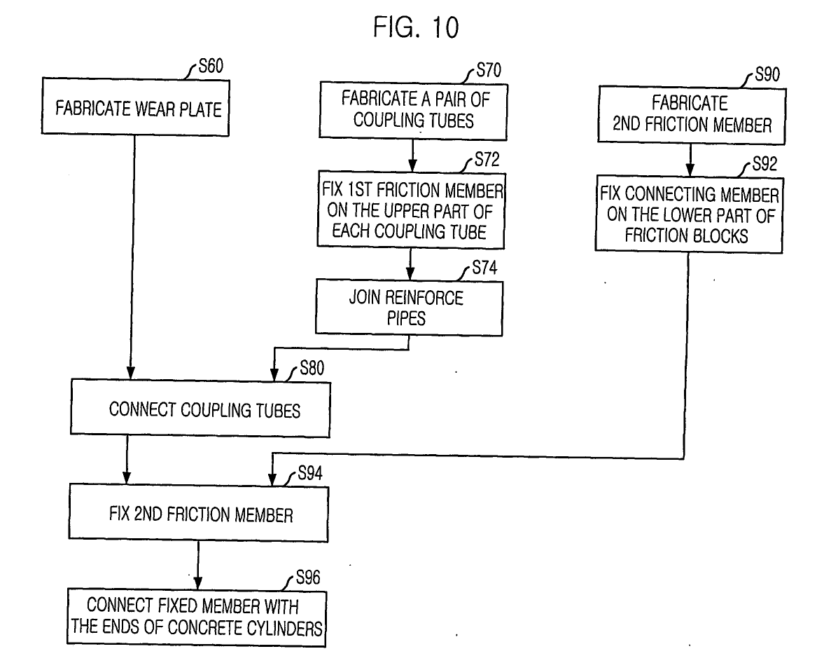

With reference to FIG. 10, the operation of a sliding motion structure having the above described elements includes the steps of fabricating the wear plate (S60), fabricating coupling tubes (S70), fixing the fist friction member (S72), connecting the coupling tubes (S80), fabricating the second friction member (S90), connecting the connecting member (S92), fixing the second friction member (S94), and installing the fixed member (S96).

At step S60, the wear plate 310 connected with a pair of concrete cylinders is fabricated. A pair of throughhole portions 312 and 313 are formed to be connected with the insides of the concrete cylinders on the right and left parts of the wear plate 310 and along the circumference area of the wear plate 310, a plurality of bolt fixing holes 314 are formed to connect the wear plate 310 to the ends of the concrete cylinders. On the surface between the throughhole portions 312 and 313, a subsidence 311 is formed in a predetermined depth and the subsidence 311 has a plurality of subsided bolt fixing holes 311a.

At step S70, a pair of coupling tubes 320 and 330 are fabricated. The coupling tubes 320 and 330 are rings having inlet ports 320a and 330a in the center. They are formed in such a size that can be tightly inserted into the throughhole portions 312 and 313. The wear plate 310 has subsided fringes 312b and 313b in the upper part of the internal wall of the throughhole portions 312 and 313 and on the other hand, protruded fringes 320b and 330b is formed in the upper part of the external wall of the coupling tubes 320 and 330 to be tightly inserted into the subsided fringes 312b and 313b. The coupling tubes 320 and 330 can be inserted tightly into predetermined positions of the throughhole portions 312 and 313 from the lower part of the wear plate 310 by inserting the protruded fringes 320b and 330b into the subsided fringes 312b and 313b.

After the step S70, a step S72 for fixing the first friction member is executed. At step S72, first friction members 325 and 335 are firmly fixed on the upper part of the coupling tubes 320 and 330. The first friction members 325 and 335, which are formed of tungsten carbide, are fabricated in the form of a plurality of friction blocks and welded along the upper part of the coupling tubes 320 and 330.

Subsequently, at step S80, the coupling tubes 320 and 330 combined with the first friction members 325 and 335 are connected with the throughhole portions 312 and 313 of the wear plate 310. The coupling tubes 320 and 330 combined with the first friction members 325 and 335 are inserted in the throughhole portions 312 and 313 in the method of shrink fitting and the coupling tubes 320 and 330 have the subsided fringes 312b and 313b in the external upper part. Also, the throughhole portions 312 and 313 of the wear plate 310 have protruded fringes 320b and 330b in the internal upper part. The coupling tubes 320 and 330 are inserted into the throughhole portions 312 and 313, until the subsided fringes 312b and 313b contact the protruded fringes 320b and 330b tightly. The first friction members 325 and 335 combined with the upper part of the coupling tubes 320 and 330 are fabricated to be protruded out from the surface of the wear plate 310 in a predetermined length, when the coupling tubes 320 and 330 are connected with the wear plate 310. Also, to enhance the connection between the coupling tubes 320 and 330 and the throughhole portions 312 and 313 of the wear plate 310, it is desirable to fix bolts along the connection between the wear plate 310 and the coupling tubes 320 and 330, which is illustrated in FIG. 9.

At step S90, a second friction member 345 is fabricated to be filled in the subsidence 311 between the throughhole portions 312 and 313 of the wear plate 310 and the second friction member 345 is formed of tungsten carbide and it is fabricated in the form of friction blocks to prevent thermal deformation. Subsequently, at step S92, a connecting member 346 is welded on the lower part of the friction blocks. When the second friction member 345 combined with the connecting member 346 is put into the subsidence 311, the upper surface of the second friction member 345 is on the same horizontal plane as that of the first friction members 325 and 335.

At step S94, the second friction member 345 combined with the connecting member 346 is fixed in the subsidence 311 firmly by utilizing bolts 343 and the bolts 343 penetrate the subsided bolt fixing holes 311a and they are connected with the connecting member 346, the second friction member 345 maintaining the same plane with the first friction members 325 and 335.

At step S96 wherein the wear plate 310, which is combined with the first and second friction members 325, 335 and 345, the coupling tubes 320 and 330, and the connecting member 346, is fixed on the ends of the concrete cylinders, a plurality of bolts penetrates the bolt fixing holes on the fringe part of the ends of the concrete cylinders and, thus, the wear plate 310 is firmly connected with the ends of the concrete cylinders by fixing in the bolt fixing holes 314 of the wear plate 310.

Between the steps S72 and S80, a step S35 for shrink-fitting reinforce members 327 and 337 can be inserted.

With a concrete pump having the fixed member 300 and the movable member in this embodiment, the same effects as the previous embodiment can be obtained.

The sliding motion structure for a concrete pump and a manufacturing method suggested in the present invention can reduce the extent of abrasion of the fixed member and the movable member remarkably. Therefore, the fixed member and the movable member can be used for a long time. This can also remove the inconvenience caused by frequent replacement of a fixed member and a movable member and solve the problem of excessive maintenance cost.

Also, when the friction members need to be replaced, the wasteful aspect of disposing the fixed member and the movable member is minimized by replacing only those worn out among friction blocks constituting each friction member. Therefore, the sliding motion structure can be maintained and repaired in an easy way at a low cost.

Moreover, since the friction members are made of a plurality of friction blocks, the sliding motion structure can be prevented from being deformed. The abrasion of the internal wall of the inlet ports of the fixed member and the internal wall of the outlet ports of the movable member can be reduced by inserting reinforce members thereto. If the third friction member of the movable member is fabricated to have a cross section of a “ [symbol] ” shape, the partial abrasion on the external end of the third friction member, which is caused by shaking movement, can be prevented.

While the present invention has been described with respect to certain preferred embodiments, it will be apparent to those skilled in the art that various changes and modifications may be made without departing from the scope of the invention as defined in the following claims.

What is claimed is:

1. A sliding motion structure for a concrete pump which extrudes concrete by using concrete pistons moving back and forth in a pair of concrete cylinders and discharges the extruded concrete through a shaking S-shaped gate valve, comprising:
a plane fixed member including a wear plate which is connected to the ends of the concrete cylinders and has a pair of throughhole portions connected to the insides of the concrete cylinders; a pair of coupling tubes which are connected with the wear plate along the throughhole portions and have inlet ports; first friction members which are formed of tungsten carbide and connected to the upper surface of the coupling tubes; and a second friction member which is formed of tungsten carbide and connected on the upper surface of the wear plate between the throughhole portions in the same height as the first friction members; and
a ring-shaped movable member including a ring-shaped connecting pipe which is connected to the ends of the S-shaped gate valve and a third friction member which is formed of tungsten carbide and connected to the first and second friction members tightly along the ends of the connecting pipe in the same length.

2. The sliding motion structure as recited in claim 1, wherein the coupling tubes of the fixed member are provided with a protrusion that is formed downwards along the internal circumference area of the lower part of the coupling tubes, the protrusion guiding the coupling tubes to the upper part of the throughhole portions of the wear plates.

3. The sliding motion structure as recited in claim 1, wherein the coupling tubes of the fixed member are provided with a standard protrusion that is formed upwards along the external circumference area of the upper part of the coupling tubes, the standard protrusion guiding the connection between the coupling tubes and the first friction members.

4. The sliding motion structure as recited in claim 1, wherein reinforce members are inserted to the internal walls of the coupling tubes of the fixed member and the internal wall of the connecting pipe of the movable member.

5. The sliding motion structure as recited in claim 4, wherein the coupling tubes of the fixed member are provided with catching protrusions formed inwards horizontally along the internal circumference of the upper part of the coupling tubes, the catching protrusions limiting the upward insertion of the reinforce members.

6. The sliding motion structure as recited in claim 1, wherein the wear plate of the fixed member is connected to the ends of the concrete cylinders by a holder which is fixed by bolts on the ends of the concrete cylinders and holding the circumference area of the wear plate.

7. The sliding motion structure as recited in claim 1, wherein the wear plate of the fixed member is connected to the ends of the concrete cylinders by bolts which are fixed on bolt fixing holes penetrating the circumference of the wear plate.

8. The sliding motion structure as recited in claim 1, wherein a connecting member is connected to the lower part of the second friction member; and the second friction member with the connecting member is connected with the wear plate by utilizing a plurality of bolts penetrating the wear plate and fixed to the connecting member.

9. The sliding motion structure as recited in claim 1, wherein a connecting member is connected to the lower part of the second friction member and a subsidence is formed on the surface of the wear plate between the first friction member s; and the second friction member with the connecting member is connected with the wear plate by utilizing a plurality of bolts penetrating the subsidence of the wear plate and fixed to the connecting member.

10. The sliding motion structure as recited in claim 1, wherein the external end of the third friction member is bent downwards in the form of embracing the friction surface and external surface of the connecting pipe so that the third friction member has a cross section of a “ [symbol] ” shape.

11. The sliding motion structure as recited in claim 1, wherein the friction members are formed of combinations of friction blocks.

12. The sliding motion structure as recited in claim 1, wherein the tungsten carbide that forms the friction members is wear-resistant alloy containing 80 to 90% WC, 2 to 25% TiC, 5 to 10% TaC and 3 to 10% TaNBC.

13. A method for manufacturing a sliding motion structure for a concrete pump, comprising the steps of:
a) fabricating a wear plate with a pair of throughhole portions connected to the insides of a pair of concrete cylinder;
b) fabricating coupling tubes having inlet ports to be tightly inserted to the throughhole portions of the wear plate;
c) connecting first friction members which are formed of tungsten carbide to the upper part of the coupling tubes;
d) connecting the coupling tubes combined with the first friction members to the throughhole portions of the wear plate, the first friction members being protruded to the upper part of the wear plate;
e) fabricating a second friction member with tungsten carbide and inserting the second friction member between the first friction members in the wear plate;
f) connecting a connecting member to the lower part of the second friction member, the upper surfaces of the first and second friction members are on the same plane; and
g) connecting the connecting member combined with the second friction member to a part of the wear plate between the first friction members, the upper surfaces of the first and second friction members are on the same plane.

14. The method as recited in claim 13, further comprising a step h) of connecting the wear plate combined with the first and second friction members to the ends of the concrete cylinders after the step g).

15. The method as recited in claim 13, further comprising a step i) of inserting reinforce members into the coupling tubes and fixing the reinforce members tightly on the internal wall surface of the coupling tubes, the step i) performed between the steps c) and d).
